(19) United States
(12) Patent Application Publication
(10) Pub. No.: US 2016/0157838 A1
(43) Pub. Date: Jun. 9, 2016

(54) CONTROLLED TRANSFER BIOLOGICAL SAMPLE COLLECTION DEVICES AND METHODS OF USING SUCH DEVICES

(71) Applicant: GE HEALTHCARE UK LIMITED, Little Chalfont, Buckinghamshire (GB)

(72) Inventors: Peter James TATNELL, Caerphilly (GB); Steven Paul TORTORELLA, Wells, ME (US); Geraint SEYMOUR, Cardiff (GB); Cheryl Louise POTTS, Pontypool (GB); Alan Stuart PIERCE, Cardiff (GB); Samantha Jane OGDEN, Cardiff (GB); Neil John WILLIAMS, Cardiff (GB)

(21) Appl. No.: 14/900,818

(22) PCT Filed: Jun. 25, 2014
(86) PCT No.: PCT/EP2014/063456

§ 371 Date: Dec. 22, 2015

(30) Foreign Application Priority Data
Jun. 28, 2013 (GB) 1311681.9

(51) Int. Cl. A61B 10/00; B01L 3/00

(52) U.S. Cl. CPC A61B 10/0096; B01L 3/508; B01L 2200/141; B01L 2300/04; B01L 2300/0609

(57) ABSTRACT

A controlled transfer biological sample material collection device is disclosed comprising: a body 12 housing a sample storage medium (30 FIG. 2a) for generally dry storage of the biological material, the body including a lid portion 40 operable to provide at least open and closed positions for generally respectively exposing or covering said storage medium; and a sample collection member 20 supported on the body for collecting the biological sample material, the collection member being pivotable from a first exposed position where collection of a biological sample is possible (FIG. 1a), to a second transfer position (FIG. 3a) which effects transfer of at least a portion of the collected sample onto said medium, the device being characterised in that the lid portion includes a receiver (44 FIG. 6a) for holding the sample collector when said lid is in the closed position.

### BACKGROUND OF THE INVENTION

1. Field of the Invention

The field of the present invention relates to a controlled transfer biological collection device using a dry solid transfer and storage medium, and a method for the collection of biological material of interest, for example genetic or proteinaceous material, in a form suitable for temporary or long term storage, and/or subsequent analysis. Specifically, the present invention provides for a sampling device that controls the transfer of the biological sample to the storage medium by holding the storage medium and a moveable sample collection member having an analyte collection surface.

2. Description of the Related Art

The collection of biological samples such as blood and extracting DNA for genetic analysis from the sample has been widely used by the forensics and medical community for identification purposes, for paternity testing, for genetic diagnostic testing in new born screening programs, for genetic typing, for assessing a predisposition to disease and for genetic characterisation for drug susceptibility. However, due to the invasive nature of blood collection, alternative non-invasive methods are coming into favour. Current methods involve scraping cellular mucosa from inside the oral cavity using any of a number of different devices such as cytobrushes, cotton or artificial fibre swabs, mouthwash swish and rinse methods, foam tipped swabs, and supported cellulosic filter paper collection techniques (known as the Bode method). These methods require time-consuming, labour intensive extraction methods.

The introduction of treated storage media into the forensics community has significantly streamlined the collection and extraction of DNA from a variety of samples. One such treated medium is available commercially under the brand name FTA® from Whatman, Inc. and is described in one or more of the following patents U.S. Pat. No. 6,627,226, U.S. Pat. No. 6,447,804, U.S. Pat. No. 6,294,203, U.S. Pat. No. 6,168,922, U.S. Pat. No. 5,976,572, U.S. Pat. No. 5,972,386, U.S. Pat. No. 5,939,259, and U.S. Pat. No. 5,756,126. The medium is used with a plastics collecting device known as Easicollect® from Whatman Inc, and described in US20100106057 (Harvey et al). This known collecting device includes an arm having buccal cell collector foam pad at one end, which arm is manipulated to collect buccal cells, and is further manipulated to pivot, and thereby to transfer those cells from the foam pad onto an FTA medium held at an opposing end of the device.

Whilst this technique is adequate, the transfer buccal cells to the treated medium in a consistent and reproducible manner remains a matter of operator skill, which is not ideal particularly where operators may seldom use the device. The correct pressure and timing of the transfer step are important, and it is essential that the exposed medium is not contaminated while transfer takes place.

Improvements in the device design were disclosed in WO2012/163788 (GE Healthcare), however, the inventors have realised that yet further improvements in the ease of use and prevention of contamination are possible.

Another similar device is described in U.S. Pat. No. 7,776,616 (Fitzco).

### BRIEF SUMMARY OF THE INVENTION

The present invention relates to a controlled transfer biological collection device using a dry solid storage and transfer medium and a method for the collection of biological material of interest—a sample (which may be genetic or proteinaceous material) in a form suitable for storage and/or subsequent analysis.

According to a first aspect, the invention provides a controlled transfer biological sample material collection device, comprising: a body housing a sample storage medium for generally dry storage of the biological material, the body including an lid portion operable to provide at least open and closed positions for generally respectively exposing or covering said storage medium; and a sample collection member supported on the body for collecting the biological sample material, the collection member being pivotable from an first exposed position where collection of a biological sample is possible, to a second transfer position which effects transfer of at least a portion of the collected sample onto said medium, the device being characterised in that the lid portion includes a receiver for holding the sample collector when said lid is in the closed position.

In an embodiment, the lid has an inner side which faces the medium, and an outer side which faces away from the medium, and the receiver is formed on the outer side.

In an embodiment, the receiver includes resilient formations for holding the sample collector.

In an embodiment, said sample storage medium is mounted to a card formation, and said card formation is slideable in the body for retrieval following said transfer.

In an embodiment, the body, the lid and the collection member are each formed from a single plastics moulding and are connected by reduced thickness areas which allow their respective pivoting.

According to a second aspect, the invention provides a method for controlled transfer biological sample material collection, the method including the steps of: i) providing a controlled transfer biological sample material collection device, comprising: a body housing a sample storage medium for generally dry storage of the biological material, the body including an lid portion operable to provide at least open and closed positions for generally respectively exposing or covering said storage medium; and a sample collection member supported on the body for collecting the biological sample material, the collection member being pivotable from a first exposed position where collection of a biological sample is possible, to a second transfer position which effects transfer of at least a portion of the collected sample onto said medium, the device being characterised in that the lid portion includes a receiver for holding the sample collector when said lid is in the closed position; ii) exposing the collection member at the first position and collecting a biological sample on the collection member; iii) moving the collection member from the first position to the second position to effect transfer of at least a portion of the collected sample to said medium; and iv) docking the sample collector in the receiver after closing the lid.

The invention extends to a device or method substantially as described herein with reference to the drawings, or any combination of features described herein. The invention is further characterised by the claims.

### BRIEF DESCRIPTION OF THE DRAWINGS

FIG. 1a is a perspective view of a preferred embodiment of the present invention showing the claimed device in an initial position;

FIG. 1b is a plan view of the device shown in FIG. 1;

FIG. 1c is a section through the device along line A-A shown in FIG. 1b;

FIGS. 2a through to FIG. 8c are further views of the device of FIG. 1a, in different positions.

### DETAILED DESCRIPTION OF THE INVENTION

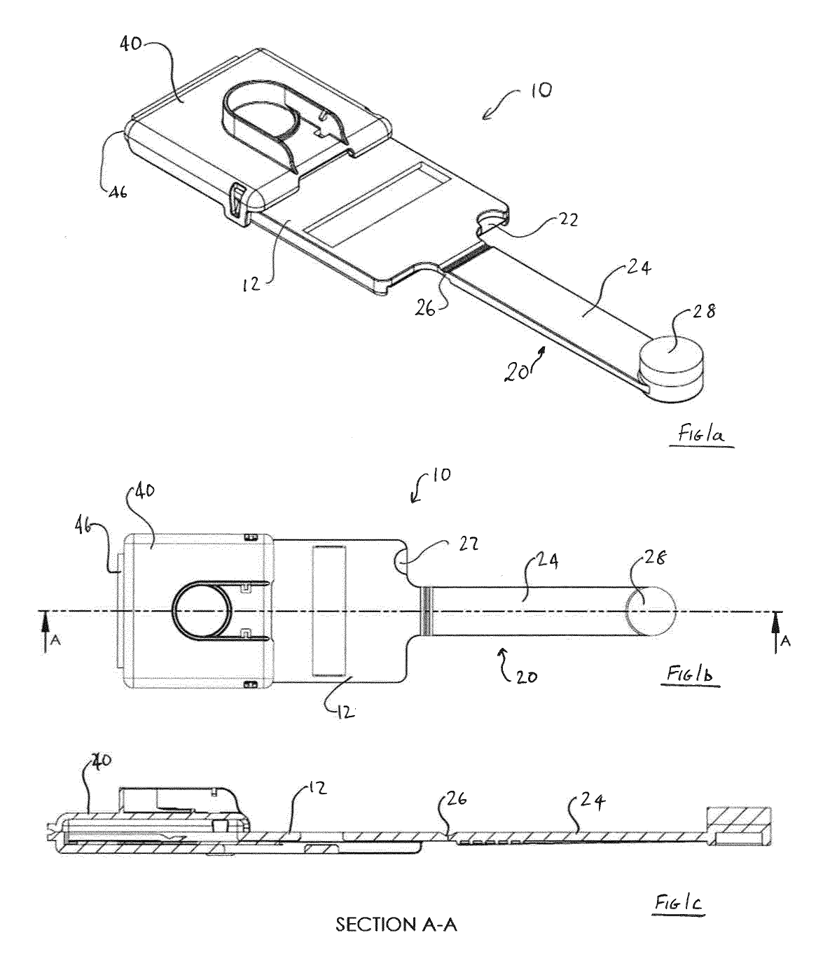

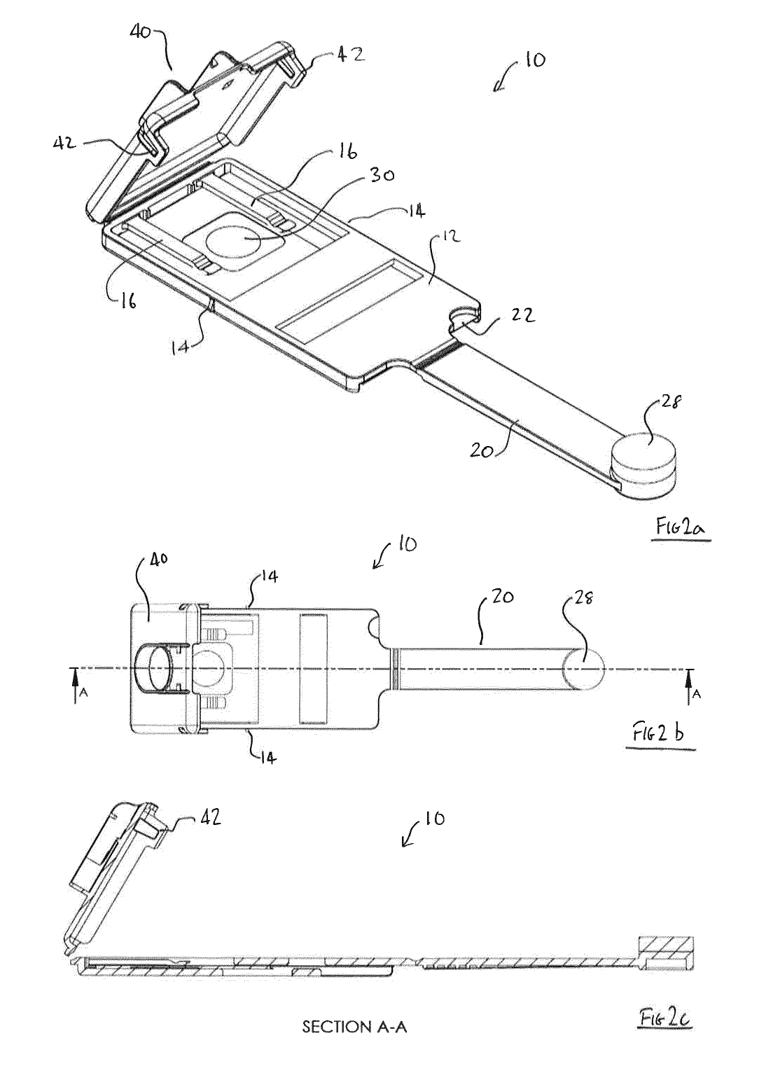

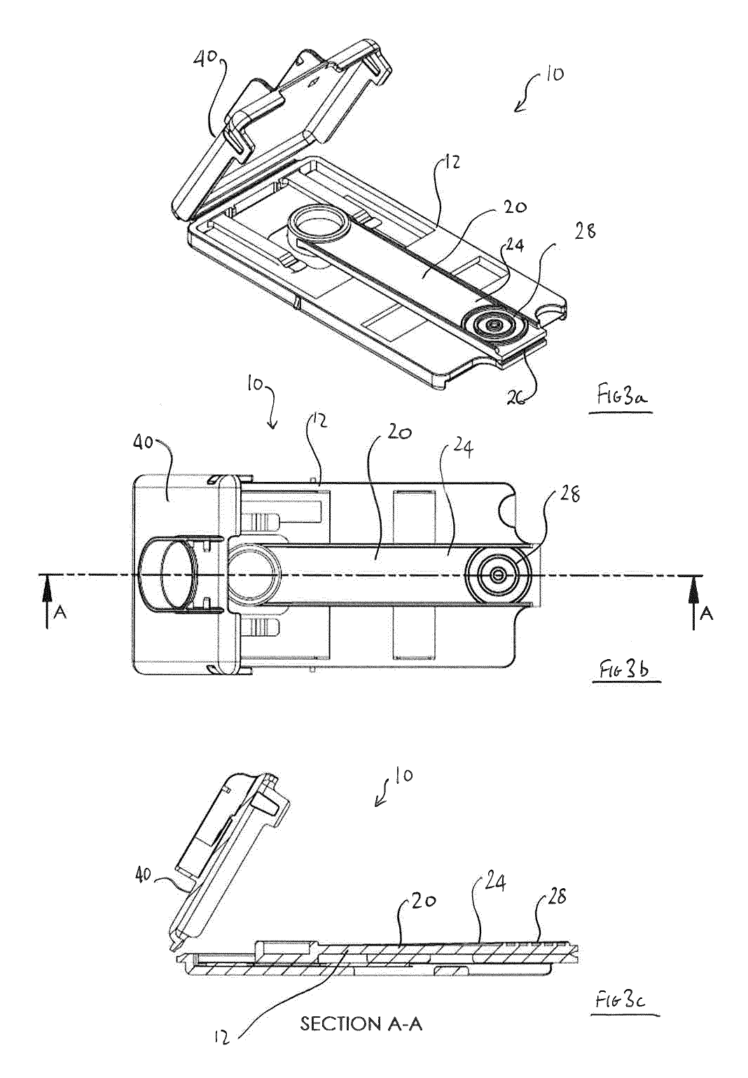

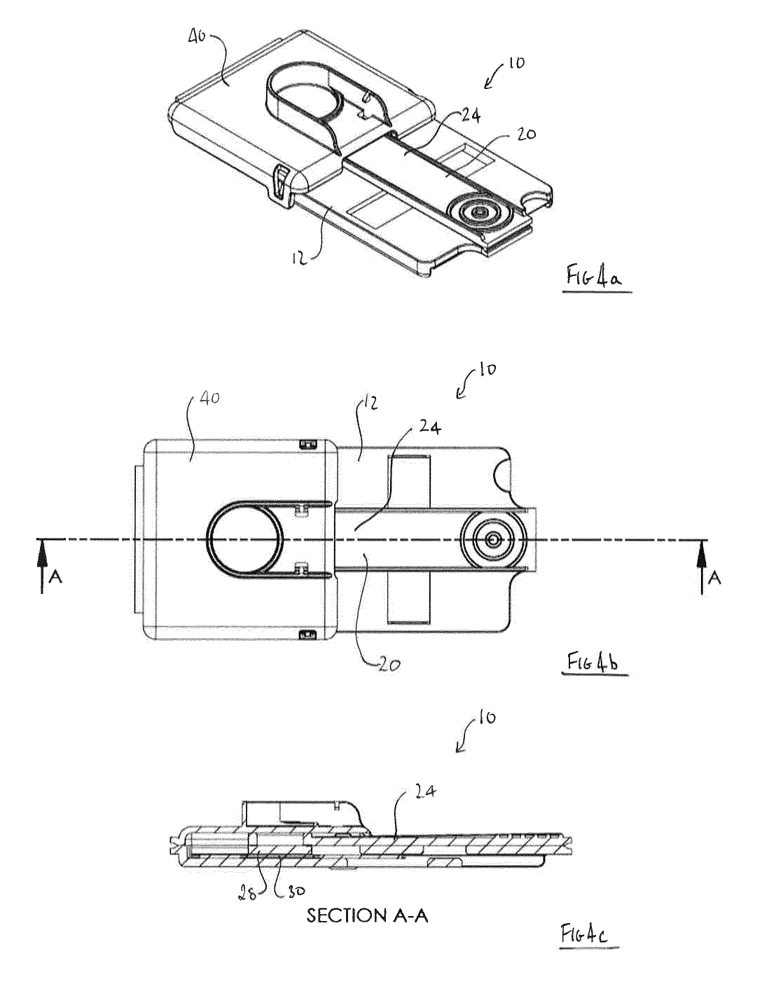

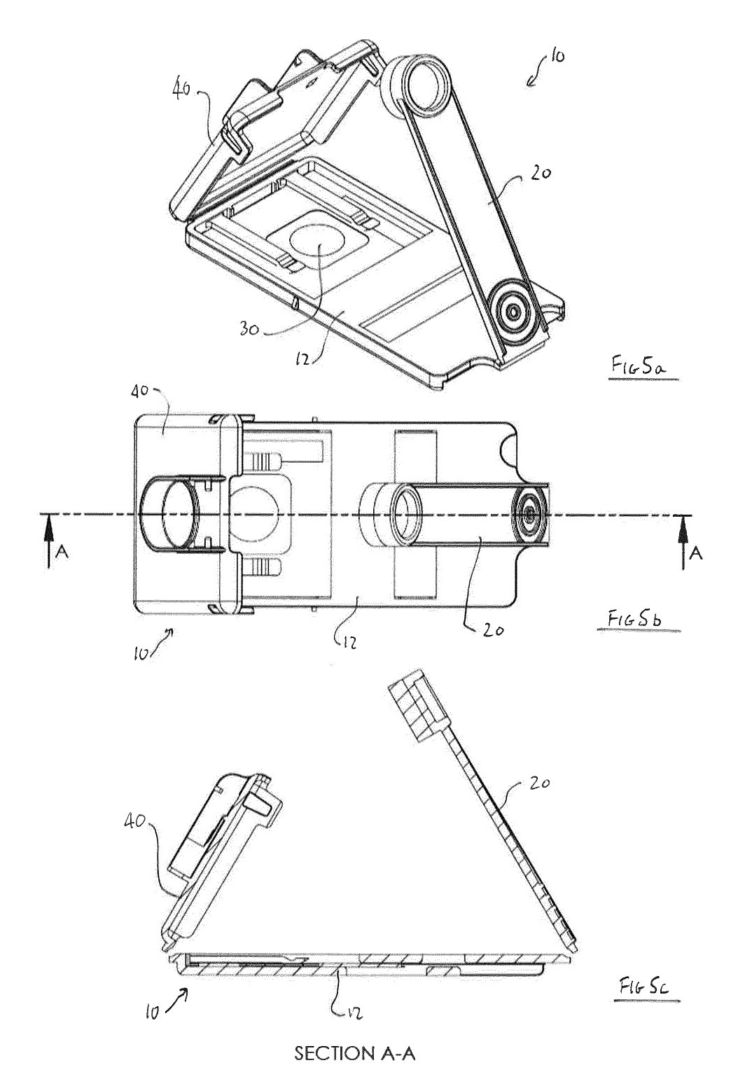

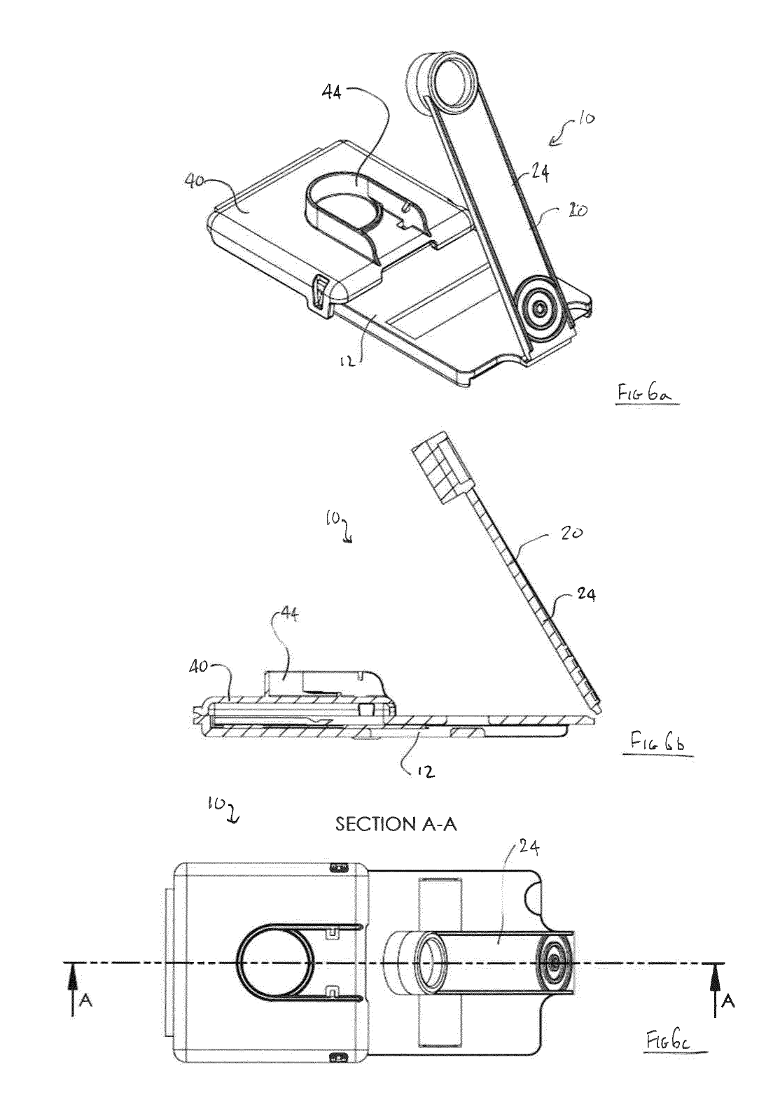

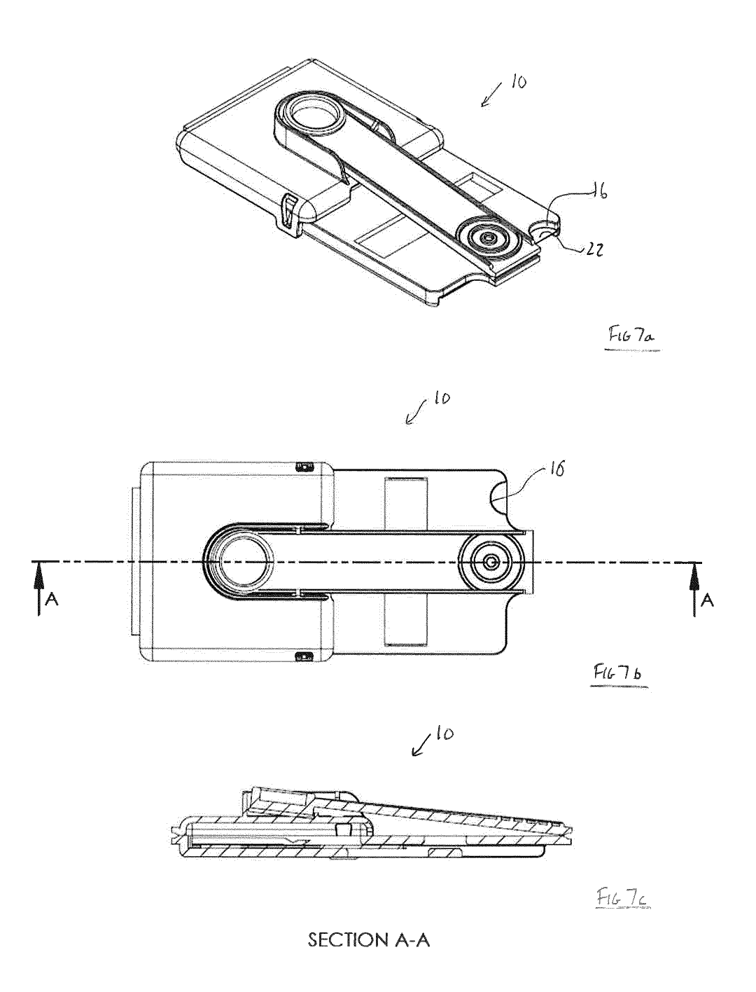

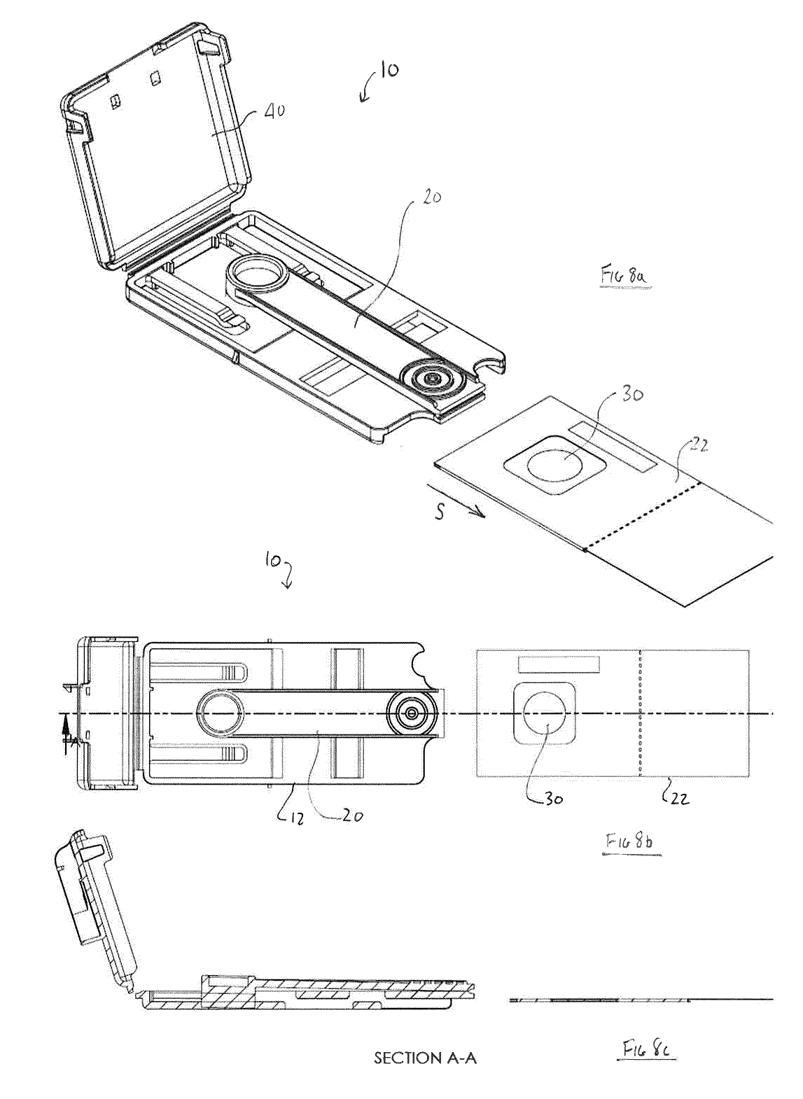

A preferred embodiment is shown in FIG. 1a. A collection device (10) for a biological sample that contains degradable biologically sourced analytes is shown which comprises a plastics moulded body 12 which pivotably supports a collection member 20 at a reduced thickness area 26, which forms a hinge. The collection member has a sample storage card 22 slideably mounted within the body 12. The card 22 has a sample storage medium (30 FIG. 2a) attached thereto. Examples of a storage medium material suitable for the present invention include untreated filter paper, such as #903® brand paper (Whatman, Inc., Florham Park, N.J. USA) or treated filter papers, such as FTA and FTA Elute brand paper (also from Whatman, Inc., Florham Park, New Jersey USA). These treated media are described in US patents referenced above. Such treated media provide a simple safe method for collection, shipping and storage of biological samples. They also contain chemistries which make it easy to isolate DNA from complex samples such as blood. Samples collected on treated or untreated media are dried for storage and can be stored at room temperature for long periods of time.

The collection member 20 includes an arm 24. At the distal end of arm 24 a resilient pad 28 of foamed polymeric material is fixed to the arm, which acts as a sample collector when required.

The collection device further includes a lid portion 40, which as will be explained below can be hinged away from the body 12, by means of pivoting the lid about a further reduced thickness portion 46 which forms a further hinge. The lid has an inner side which faces the medium 30, and an outer side which faces away from the medium. The reduced thicknesses 26 and 46 are disposed at opposite ends of the body 12.

In the position shown in FIGS. 1a and 1b, the device is ready to be used to collect a biological sample by wiping the pad 28, or otherwise making contact, over an area of interest, for example to collect buccal cells from the inner cheek surface of the mouth of a subject.

FIG. 1b shows the device 10 in plan view.

FIG. 1c shows the device 10 in section along line A-A in FIG. 1b.

Once the sample has been collected, the lid 40 is lifted away from the body 12 to reveal the sample storage medium 30, as illustrated in FIG. 2a. This is achieved by manipulating the lid 40 to overcome the retaining forces provided by a pair of retaining clips 42 which cooperate with complementary formations 14 on the body. As can be seen in FIG. 2a, the card 22 is resiliently held in place in the body 12 by a pair of sprung fingers 16.

FIG. 2b shows a plan view of the device in the position captured in FIG. 2a, and FIG. 2c is a sectional view on line A-A in FIG. 2b.

Following the lifting of the lid 40, the arm 20 is pivoted over toward the medium 30 as illustrated in FIGS. 3a, 3b, and 3c. In this position, the pad 28 may lightly touch the medium 30 to transfer some biological sample material to the medium but that is not certain. A thumb grip 28 on the arm 24 aids the user in the act of pivoting. A shown in FIGS. 4a, 4b and 4c, the user then pushes the lid 40 onto the back of the arm 24 to force the pad 28 onto the medium 30 for a predefined period on time, and thereby a controlled transfer of biological material onto the medium 30 is obtained.

Following this transfer step, the lid 40 is opened and the arm 24 is pivoted away from the medium 30, as shown in FIGS. 5a, 5b and 5c. Following this step, the lid is again closed, as shown in FIGS. 6a, 6b and 6c. The arm 24 is then stowed by docking the 10 end of the arm, including the pad 28, into a receiver 44 formed on the outer side (the back) of the lid, as shown in FIGS. 7a, 7b and 7c. The arm is held in the receiver 44 by means of resilient clip formations 46 (FIG. 6c).

Finally, the storage medium can be accessed by sliding the card 22 from the body 12 in the direction of arrow S, as illustrated in FIGS. 8a, 8b and 8c.

Since the medium 30 may contain at least one stabilizing reagent the sample will then be preserved for transport or storage. Suitable such reagents include either the combination of a weak base, a chelating agent, and, optionally, uric acid or a urate salt or simply the addition of a chaotropic salt, alone or in combination with a surfactant. The “weak base” of the composition may be a Lewis base which has a pH of about 6 to 10, preferably about pH 8 to 9.5. One function of the weak base is to act as a buffer to maintain a composition pH of about 6 to 10, preferably about pH 8.0 to 9.5, for example, pH 8.6. Hence, a weak base suitable for the composition of the invention may, in conjunction with other components of the composition, provide a composition pH of 6 to 10, preferably, about pH 8.0 to 9.5. Suitable weak bases according to the invention include organic and inorganic bases. Suitable inorganic weak bases include, for example, an alkali metal carbonate, bicarbonate, phosphate or borate (e.g., sodium, lithium, or potassium carbonate). Suitable organic weak bases include, for example, tris-hydroxymethyl amino methane (Tris), ethanolamine, tri-ethanolamine and glycine and alkaline salts of organic acids (e.g., trisodium citrate). A preferred organic weak base is a weak monovalent organic base, for example, Tris. The Tris may be either a free base or a salt, for example, a carbonate salt.

A preferred chelating agent is a strong chelating agent. By “strong” chelating agent it is meant that the agent binds multivalent metal ions with a comparable or better affinity than ethylene diamine tetraacetic acid (EDTA). A preferred chelating agent according to the invention is EDTA.

Anioinic surfactants are examples of surfactants which are useful in the present invention. A preferred anionic detergent is a strong anionic detergent. As used herein, a “strong” anionic detergent includes a hydrocarbon moiety, aliphatic or aromatic, containing one or more anionic groups. Particularly preferred anionic detergents suitable for the invention include sodium dodecyl sulphate (SDS) and sodium lauryl sarcosinate (SLS). In a preferred embodiment, the anionic detergent causes inactivation of most microorganisms which have protein or lipids in their outer membranes or capsids, for example, fungi, bacteria or viruses. This includes microorganisms which may be pathogenic to humans and are present in a biological sample.

Also preferably, the storage medium will have a visual delineation (32 FIG. 6a) placed around the transfer area of the storage medium such that if removed from the tray 22 a user will know where the material was deposited without reference to the device.

The present device 10 can be used to collect samples such as degradable biologically sourced analytes such as nucleic acids, proteins, and respective fragments thereof. The biological sample can be selected from the group consisting of saliva, blood, serum, lymph fluids, buccal cells, mucosal cells, cerebrospinal fluid, semen, vaginal fluid, faeces, plasma, urine, a suspension of cells, or a suspension of cells and viruses.

Preferably, the present device is dimensioned and configured such that the body 12 releasably holds the card 22, which can be removed by grasping the card at a recess 16 (FIGS. 7a and 7b). However, automated extraction, of the card 22 is possible also.

If used in buccal cell collection, the pad 28 should be dimensioned and configured to fit within the human mouth. For record keeping and traceability the present device should also comprise an identification label (such as conventional bar coding) on not only the medium 30, but also the body 12. RFID tags may be employed for this purpose.

To ensure device integrity, the present device can also comprise a sterility envelope surrounding the other device elements. Preferably, those other elements are sterile and free from any biological sample analytes (made for example, from medical grade plastics), which can be done through conventional techniques such as irradiation after the envelope is sealed.

Kits can be made that incorporate the above device along with any combination of associated equipment or reagents including purification reagents, buffers, or the like and storage systems, containers, or the like. In this regard, the present invention further provides a kit comprising a device as defined herein and one or more components selected from the group consisting of purification reagents for subsequent analysis of the sample, buffers, storage systems and containers.

### Example of Device Use

The present device can be used for biological sample collection for the following purposes: the collection of buccal cell samples for criminal databases; the collection of crime scene samples (i.e., rehydrated blood, semen, saliva and liquid samples of the same); the collection of sexual assault samples; the collection of buccal samples for population genetics or pharmacogenomics studies; the collection of buccal samples for personal genetic ID archiving; the collection of bacterial or parasite samples from food sources; the collection of blood from meat at slaughterhouse for meat traceability; or the collection of biological samples from animals for veterinary diagnostics.

Although one embodiment only has been illustrated, it will be apparent to the skilled addressee that modifications, variants, additions and omissions are possible within the scope and spirit of the invention defined herein.

For example, moulded plastics is the preferred material for the body 12 and collection member 20, but other materials could be employed, for example metals, for example diecast aluminium alloy.

1. A controlled transfer biological sample material collection device, comprising: a body housing a sample storage medium for generally dry storage of the biological material, the body including an lid portion operable to provide at least open and closed positions for generally respectively exposing or covering said storage medium; and a sample collection member supported on the body for collecting the biological sample material, the collection member being pivotable from a first exposed position where collection of a biological sample is possible, to a second transfer position which effects transfer of at least a portion of the collected sample onto said medium, the device being characterised in that the lid portion includes a receiver for holding the sample collector when said lid is in the closed position.

2. A device as claimed in claim 1, wherein the lid has an inner side which faces the medium, and an outer side which faces away from the medium, and the receiver is formed on the outer side.

3. A device as claimed in claim 2, wherein the receiver includes resilient formations for holding the sample collector.

4. A device as claimed in claim 3, wherein said sample storage medium is mounted to a card formation, and said card formation is slideable in the body for retrieval following said transfer.

5. A device as claimed in any one of the preceding claims, wherein the body, the lid and the collection member are each formed from a single plastics moulding and are connected by reduced thickness areas which allow their respective pivoting.

6. A method for controlled transfer biological sample material collection, the method including the steps of:
i) providing a controlled transfer biological sample material collection device, comprising: a body housing a sample storage medium for generally dry storage of the biological material, the body including an lid portion operable to provide at least open and closed positions for generally respectively exposing or covering said storage medium; and a sample collection member supported on the body for collecting the biological sample material, the collection member being pivotable from a first exposed position where collection of a biological sample is possible, to a second transfer position which effects transfer of at least a portion of the collected sample onto said medium, the device being characterised in that the lid portion includes a receiver for holding the sample collector when said lid is in the closed position;
ii) exposing the collection member at the first position and collecting a biological sample on the collection member;
iii) moving the collection member from the first position to the second position to effect transfer of at least a portion of the collected sample to said medium; and
iv) docking the sample collector in the receiver after closing the lid.

7. A device or method substantially as described herein with reference to the drawings.
